(19) United States
(12) Patent Application Publication
(10) Pub. No.: US 2020/0023148 A1
(43) Pub. Date: Jan. 23, 2020

(54) OPTICAL DRY POWDER INHALER DOSE SENSOR

(71) Applicant: MICRODOSE THERAPEUTX, INC., Ewing, NJ (US)

(72) Inventors: Douglas WEITZEL, Hamilton, NJ (US); Philip CHAN, Skillman, NJ (US)

(73) Assignee: MICRODOSE THERAPEUTX, INC., Ewing, NJ (US)

(21) Appl. No.: 16/496,052

(22) PCT Filed: Mar. 21, 2018
(86) PCT No.: PCT/US2018/023562

§ 371 Date: Sep. 20, 2019

Related U.S. Application Data
(60) Provisional application No. 62/475,095, filed on Mar. 22, 2017.

(51) Int. Cl. A61M 15/00

(52) U.S. Cl. CPC A61M 15/008; A61M 15/0005; A61M 15/009; A61M 15/0051; A61M 15/0085; A61M 2205/3334; A61M 2205/3313; A61M 2205/3331; A61M 2230/42; A61M 2205/0294; A61M 2205/0211; A61M 2202/064

(57) ABSTRACT

A dry powder inhaler including a first chamber having an orifice for holding a dry powder and a gas, and a second chamber connected to the first chamber by at least one passageway for receiving an aerosolized form of the dry powder from the first chamber and delivering the aerosolized dry powder to a user. At least one optical sensor monitors aerosolized powder particles passing in the second chamber. A vibrator coupled to the first chamber aerosolizes the dry powder and causes the aerosolized powder to move through the at least one passageway thereby delivering the powder from the first chamber to the second chamber as an aerosolized dry powder. A vibrator control unit controls operation of the vibrator based on the amount of aerosolized powder particles passing in the second chamber and delivered to a user.

### CROSS-REFERENCE TO PRIOR APPLICATIONS

This application claims priority to U.S. Provisional Patent Application No. 62/475,095, filed Mar. 22, 2017, which is hereby expressly incorporated by reference in its entirety.

### FIELD

The embodiments described herein relate generally to the field of the delivery of pharmaceuticals and drugs. Particular utility may be found in monitoring and regulating the delivery of a pharmaceutical or drug to a patient and will be described in connection with such utility, although other utilities are contemplated.

### BACKGROUND

Certain diseases of the respiratory tract are known to respond to treatment by the direct application of therapeutic agents. As these agents are most readily available in dry powdered form, their application is most conveniently accomplished by inhaling the powdered material through the nose or mouth. This powdered form results in the better utilization of the medication in that the drug is deposited exactly at the site desired and where its action may be required; hence, very minute doses of the drug are often equally as efficacious as larger doses administered by other means, with a consequent marked reduction in the incidence of undesired side effects and medication cost. Alternatively, the drug in powdered form may be used for treatment of diseases other than those of the respiratory system. When the drug is deposited on the very large surface areas of the lungs, it may be very rapidly absorbed into the blood stream; hence, this method of application may take the place of administration by injection, tablet, or other conventional means.

Current dry powder inhalers (DPIs), generally being passive devices, contain no sensor or mechanism to confirm that a dose of the dry powder formulation has been successfully delivered to the patient. Depending on the method used by the DPI for metering and dispensing the formulation, there are a variety of failure modes that can prevent successful delivery of a complete dose to the user. Among these failure modes are: (1) mechanical failure of formulation metering mechanism preventing the proper amount of formulation from being presented to the inhalation channel; (2) clogging of internal channels or de-aggregation meshes due to powder build-up, especially if moisture is introduced into the inhaler, such that formulation cannot flow freely as intended; (3) failure of capsule piercing mechanisms preventing powder from getting out of the primary drug packaging; (4) failure of blister strip materials (such as peelable lidding), peeling mechanisms or dose advance mechanisms preventing powder from getting out of the primary drug packaging; and (5) patient-related failure modes, such as insufficient inspiratory flow or exhaling into an inhaler.

While inhaler dose counters can indicate that an inhaler was properly actuated, the dose counter mechanism cannot confirm that formulation was properly delivered via inhalation to the user. In some cases, the patient may detect a taste associated with the drug formulation, but this method is unreliable because it depends on the specific formulation being delivered or the patients' sense of taste, which can be affected by a number of factors including food or drink taken just prior to using the inhaler or the presence of certain symptoms of illness, such as nasal congestion or inflammation of oral, dental or lingual tissue that could adversely affect taste. Furthermore, in high efficiency active DPI devices, smaller amounts of formulation may be delivered more directly to the respiratory tract without sticking to the inside of the mouth or tongue, in which case insufficient amounts of material may be present in the mouth to be detected through the sense of taste.

### SUMMARY OF THE INVENTION

Embodiments described herein relate to methods, apparatuses, and/or systems for regulating the dosage of a pharmaceutical(s) or drug(s) delivered through an inhaler. In certain embodiments, the inhaler is capable of detecting that the drug or medication was delivered in the correct amount and under the correct conditions (such as inspiratory flow) to the user. In some embodiments, this information is clearly presented to the user immediately after taking a dose with the inhaler.

These methods, apparatuses, and/or systems provide significant advantages over known DPIs. Products and instruments used for sensing the presence and/or flow of particulate matter are currently available for a large variety of applications utilizing a variety of sensing technologies. These products generally rely on technologies such as reflective or transmissive optical approaches using ambient, infrared or laser illumination; detection of electrostatic charge on moving particles; ultrasonic ranging; radio frequency/microwave Doppler flowmetry; or ionization chamber systems using radioactive materials. Most of these types of sensor systems rely on relatively expensive components and materials, often require periodic calibration, and are intended to make accurate measurements. Furthermore, the physical size of the hardware used in most of these approaches would be prohibitive for use in battery-operated, hand-held devices, and in the case of radioactive ionization chamber devices, would present a patient health or safety hazard. From among this list of technologies, however, optical sensors using infrared or visible illumination offer opportunities for very low-cost implementations, especially if a lower degree of accuracy is acceptable for the application. Specifically, the use of infrared-sensitive components is preferred because they are less sensitive to ambient light interference, the technology is mature, thus reducing technical and component availability risks, and therefore tends to be very low cost. Optical sensors are relatively unaffected by powder formulation, ambient humidity or electrical interference. Immunity to the effects of humidity is particularly important when the sensor is used in a tidal inhaler in which humid patient exhalation is present. Thus, optical sensing of the drug or medication being delivered to the user is ideal to cure the shortcoming of known DPIs mentioned above.

Various other aspects, features, and advantages of the embodiments will be apparent through the detailed description and the drawings attached hereto. It is also to be understood that both the foregoing general description and the following detailed description are exem.plary and not restrictive of the scope of the embodiments. As used in the specification and in the claims, the singular forms of “a”, “an”, and “the” include plural referents unless the context clearly dictates otherwise. In addition, as used in the specification and the claims, the term “or” means “and/or” unless the context clearly dictates otherwise. Moreover, the use of the term pharmaceutical and/or drug denotes a single active ingredient, or combinations of active ingredients and is not intended to be construed as limited to a single active ingredient. Finally, the description herein of disadvantages and shortcomings of certain known devices or methods is not intended to exclude the known devices or methods from the scope of the claims. Indeed, certain embodiments may include the use of known devices or methods, without suffering from the herein described disadvantages and shortcomings.

### BRIEF DESCRIPTION OF THE DRAWINGS

FIG. 1 shows perspective views of an inhaler, in accordance with one or more embodiments.

FIGS. 2-3 show perspective views of an optical sensor arrangement mated to a mouthpiece of the inhaler as an external apparatus, in accordance with one or more embodiments.

FIG. 4 shows an exemplary circuit diagram of an optical sensor signal conditioning circuit, in accordance with one or more embodiments.

FIG. 5 shows a functional block diagram of an inhaler controller, in accordance with one or more embodiments.

FIG. 6 shows a graph depicting an exemplary optical sensor output signal and area-under-the-curve calculated from the output signal, in accordance with one or more embodiments.

FIG. 7 shows a graph depicting an output of a particle size analyzer for a single dosing sequence, in accordance with one or more embodiments.

FIG. 8 shows a depiction of the optical dose sensor output fine particles and (b) coarse particles, in accordance with one or more embodiments.

FIG. 9 shows a graph depicting the accuracy of linear modeling versus values of weighting factors a and b, in accordance with one or more embodiments.

FIG. 10 shows a graph depicting linear regression analysis of initial calibration of a sample set using equal weighting factors under AUC and RMS, in accordance with one or more embodiments.

FIG. 11 shows a graph depicting linear regression analysis for a second calibration of a sample set including larger doses of powder, in accordance with one or more embodiments.

FIG. 12 shows a graph depicting linear regression analysis for both the initial calibration sample set and the second calibration sample set, in accordance with one or more embodiments.

FIG. 13 shows a flowchart of a method 200 of delivering a drug with an inhaler, in accordance with one or more embodiments.

### DETAILED DESCRIPTION

In the following description, for the purposes of explanation, numerous specific details are set forth in order to provide a thorough understanding of the embodiments. It will be appreciated, however, by those having skill in the art that the embodiments may be practiced without these specific details or with an equivalent arrangement. In other instances, well-known structures and devices are shown in block diagram form in order to avoid unnecessarily obscuring the embodiments of the invention.

The present embodiments relate to a device for administering medicament as a dry powder for inhalation by a subject. Some embodiments of the device may be classified as a dry powder inhaler (DPI). Some embodiments of the device may also be classified as a dry powder nebulizer (as opposed to a liquid nebulizer), particularly when tidal breathing is used to deliver dry powder medicament over multiple inhalations. The device may be referred to herein interchangeably as a “device” or an “inhaler,” both of which refer to a device for administering medicament as a dry powder for inhalation by a subject, preferably over multiple inhalations, and most preferably when tidal breathing is used. “Tidal breathing” preferably refers to inhalation and exhalation during normal breathing at rest, as opposed to forceful breathing.

Structure and Operation of an Inhalation Device

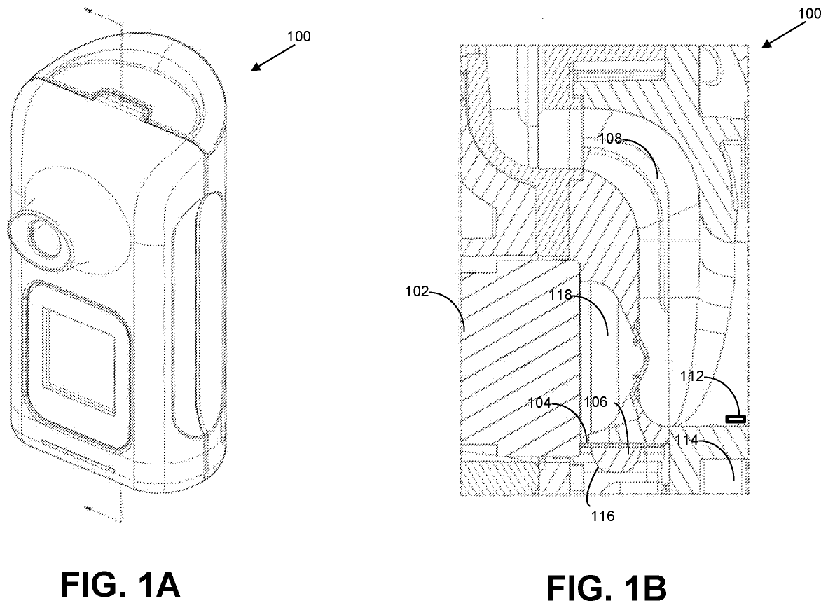

FIGS. 1A-C show an inhaler 100 configured to receive a user's inhalation through the mouthpiece of the device, preferably via tidal breathing, and deliver a dose of medicament over a plurality of consecutive inhalations. In one embodiment illustrated in FIGS. 1A-C, the inhaler 100 may be configured to activate transducer 102 more than once to deliver a complete pharmaceutical dose from a drug cartridge 104 to a user. During operation, when the user inhales through the mouthpiece, air is drawn into the inhaler's air inlet, through an air flow conduit in the device, and out of the mouthpiece into the user's lungs; as air is being inhaled through the air flow conduit, dry powder medicament is expelled into the airflow pathway and becomes entrained in the user's inhaled air. Thus, the air flow conduit preferably defines an air path from the air inlet to the outlet (i.e., the opening that is formed by the mouthpiece). Each breath cycle includes an inhalation and an exhalation, i.e., each inhalation is followed by an exhalation, so consecutive inhalations preferably refer to the inhalations in consecutive breath cycles. After each inhalation, the user may either exhale back into the mouthpiece of the inhaler, or exhale outside of the inhaler (e.g., by removing his or her mouth from the mouthpiece and expelling the inhaled air off to the side). In one embodiment, consecutive inhalations refer to each time a user inhales through the inhaler which may or may not be each time a patient inhales their breath.

In one embodiment, the inhaler 100 may contain a plurality of pre-metered doses of a dry powder drug composition comprising at least one medicament, wherein each individual dose of the plurality of pre-metered doses is inside a drug cartridge 104, such as a blister 106. As used herein, a blister 106 may include a container that is suitable for containing a dose of dry powder medicament. Preferably, a plurality of blisters may be arranged as pockets on a strip, i.e., a drug cartridge. According to a preferred embodiment, the individual blisters may be arranged on a peelable drug strip or package, which comprises a base sheet in which blisters are formed to define pockets therein for containing distinct medicament doses and a lid sheet which is sealed to the base sheet in such a manner that the lid sheet and the base sheet can be peeled apart; thus, the respective base and lid sheets are peelably separable from each other to release the dose contained inside each blister. The blisters may also be preferably arranged in a spaced fashion, more preferably in progressive arrangement series progression) on the strip such that each dose is separately accessible.

FIGS. 1A-C shows an inhaler 100 configured to activate the transducer 102 more than once to deliver a complete pharmaceutical dose from a single blister 120 to a user. In one embodiment, the inhaler 100 may include an air flow conduit 108 configured to allow air to travel through the inhaler 100 when a user inhales through a mouthpiece 110. In one embodiment, the inhaler 100 may include an inhalation sensor 112 configured to detect airflow through the air flow conduit 108 and send a signal to a controller 114 when airflow is detected. In one embodiment, the controller 114 may be configured to activate a drug strip advance mechanism 116, when a flow of air is detected by the sensor 112 (in some cases, when a first flow of air is detected). The drug strip advance mechanism 116 may be configured to advance a drug strip 104 a fixed distance (e.g., the length of one blister) such that the blister 106 is in close proximity to (or in one embodiment adjacent to or substantially adjacent to) a dosing chamber 118, for example. A membrane (not shown) may be configured to cover an open end of the dosing chamber 118 in one embodiment. In one embodiment. transducer 102 may confront the membrane of the dosing chamber 118. In one embodiment, the controller 114 may be configured to activate a transducer 102 when an activation event is detected. In one embodiment, detection of multiple inhalations may be required to trigger activation of transducer 102. For example, controller 114 may be configured to activate a transducer 102 when a flow of air is detected by the sensor 112 (in some cases, when a subsequent flow of air is detected, e.g., second, third, or later) The transducer 102 may be configured to vibrate, thereby vibrating the membrane, to aerosolize and transfer pharmaceutical from the blister 106 into the dosing chamber 118. In one embodiment, the vibration of the transducer 102 also delivers the aerosolized pharmaceutical into the dosing chamber 118, through the exit channel 120, and to a user through mouthpiece 110.

The transducer 102 may be a piezoelectric element made of a material that has a high-frequency, and preferably, ultrasonic resonant vibratory frequency (e.g., about 15 to 50 kHz), and is caused to vibrate with a particular frequency and amplitude depending upon the frequency and/or amplitude of excitation electricity applied to the piezoelectric element. Examples of materials that can be used to comprise the piezoelectric element may include quartz and polycrystalline ceramic materials (e.g., barium titanate and lead zirconate titanate). Advantageously, by vibrating the piezoelectric element at ultrasonic frequencies, the noise associated with vibrating the piezoelectric element at lower (i.e., sonic) frequencies can be avoided.

In some embodiments, the inhaler 100 may comprise an inhalation sensor 112 (also referred to herein as a flow sensor or breath sensor) that senses when a patient inhales through the device, for example, the sensor 112 may be in the form of a inhalation sensor, air stream velocity sensor or temperature sensor. According to one embodiment, an electronic signal may be transmitting to controller 114 contained in inhaler 100 each time the sensor 112 detects an inhalation by a user such that the dose is delivered over several inhalations by the user. For example, sensor 112 may comprise a conventional flow sensor which generates electronic signals indicative of the flow and/or pressure of the air stream in the air flow conduit 108, and transmits those signals via electrical connection to controller 114 contained in inhaler 100 for controlling actuation of the transducer 102 based upon those signals and a dosing scheme stored in memory (not shown). Preferably, sensor 112 may be an inhalation sensor. Non-limiting examples of inhalation sensors that may be used in accordance with embodiments may include a microelectromechanical system (MEMS) inhalation sensor or a nanoelectromechanical system (NEMS) inhalation sensor herein. The inhalation sensor may be located in or near an air flow conduit 108 to detect when a user is inhaling through the mouthpiece 110.

Inhaler 100 may also include a miniature infrared (IR) optical sensor 113 positioned on the inner surface of air flow conduit 108 to sense particles of powder medication passing by the optical sensor 113 through air stream F. Preferably, optical sensor 113 may be positioned such that the powder medication delivered into the user's inspiratory flow path passes by and is sensed by optical sensor 113. In one embodiment, optical sensor 113 may include a transmitter (light-emitting diode or LED) and receiver (phototransistor receiver) situated such that IR illumination from the transmitter is projected directly onto the receiver. In another embodiment, optical sensor 113 may comprise both an IR transmitter and receiver such that illumination from the transmitter reflects off the particles in from of the sensor are received by the receiver. Preferably, optical sensor 113 may generate signals indicating the amount of powder medication to pass through air flow conduit 108 through air stream F, and transmit those signals via, electrical connection to controller 114.

Preferably, the controller 114 may be embodied as an application specific integrated circuit chip and/or some other type of very highly integrated circuit chip. Alternatively, controller 114 may take the form of a microprocessor, or discrete electrical and electronic components. As will be described more fully below, the controller 114 may control the power supplied from conventional power source 154 (e.g., one or more D.C. batteries) to the transducer 102 according to the breathing cycle of the user and/or the amount of powder medication that has passed though air flow conduit 108 and delivered to the user. The power may be supplied to the transducer 102 via electrical connection between the vibrator and the controller 114. In one embodiment, an electrical excitation may be applied to the transducer 102 generated by the controller 114 and an electrical power conversion sub-circuit (not shown) converts the DC power supply to high-voltage pulses (typically 220 Vpk−pk) at the excitation frequency.

Memory may include non-transitory storage media that electronically stores information. The memory may include one or more of optically readable storage media, electrical charge-based storage media (e.g., EEPROM, RAM, etc.), solid-state storage media (e.g., flash drive, etc.), and/or other electronically readable storage media. The electronic storage may store dosing algorithms, information determined by the processors, information received from sensors, or other information that enables the functionality as described herein.

In operation, blister 106 may be punctured and inserted onto the membrane in dosing chamber 118 in the manner described previously. The user inhales air through the air flow conduit 108 and air stream is generated through air flow conduit 108. The flow and/or pressure of inhalation of air stream F may be sensed by a sensor 112 and transmitted to controller 114, which supplies power to transducer 102 based according to the signals and a stored dosing scheme. For example, for each inhalation detected by inhalation sensor 112, controller 114 may activate transducer 102 for a predetermined amount of time. Controller 114 may adjust the amplitude and frequency of power supplied to the transducer 102 until they are optimized for the best possible deaggregation and suspension of the powder from the capsule into the air stream via air flow. Controller 114 may also control activation of the transducer 102 according to the amount of powder medication delivered to the user based on the signals received from optical sensor 113. In sonic embodiment, controller 114 may activate transducer 102 at the start of each inhalation of the user for a series of breath cycles until all the powder medication for the dosing session has been delivered into the user's inspiratory flow. Controller 114 may also control a user interface (not shown) on the inhaler which indicates whether each dose of medication was properly taken based on the signals received from inhalation sensor 112 and/or optical sensor 113.

Optical Sensor Structure and Operation

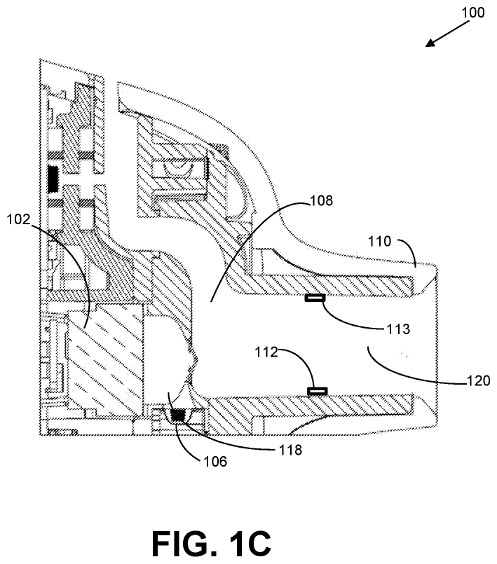

FIG. 2 shows a top view of an IR sensor tube assembly of an inhaler in accordance with an embodiment. As shown in FIG. 2, the optical sensor 113 may be positioned on the inner surface of air flow conduit 108 of inhaler 100 to sense the passing of particles of powder medication by the sensor 113 through air stream. FIG. 3 shows a top view of another IR sensor tube assembly of an inhaler in accordance with an embodiment. As shown in FIG. 3. inhaler 100 may include a mouthpiece 110 to assist in the delivery of powder medication to the user. Optical sensor 113 may be positioned on the inner surface of the air flow conduit 108 of inhaler 100, adjacent to the mouthpiece 110, to sense the passing of particles of powder medication by the sensor 113 through air stream.

It will be appreciated by those having ordinary skill in the art that optical sensor 113 may be configured for either reflective-mode or transmissive mode operation. In the reflective mode, the optical sensor 113 may comprise both an IR transmitter (light-emitting diode, or LED) and a phototransistor receiver designed for optimal response at the wavelength used by the transmitter. Both the transmitter and receiver elements may be situated within the sensor package so that illumination from the transmitter element reflected off material in front of the sensor within a certain working distance is efficiently received by the receiving element. For example, IR light transmitted by the LED may be reflected off the drug formulation particles as they travel past the line-of-sight of optical sensor 113, and can be received by the phototransistor to be converted to an electronic signal.

In the reflective mode of operation, a minimum sensor signal indicates that no formulation is present, and a maximum signal indicates that a large amount of formulation is present. Signal conditioning electronics amplify the electronic signal from the receiver to voltage levels that are compatible with controller 114, typically in the 0 to 3.3 V range. The signal conditioning electronics also supply a stable current source to the transmitter, and may also apply filtering to reduce electronic or thermal noise present in the sensor output.

In the transmissive mode of operation, a transmitter and receiver of optical sensor 113 may be situated such that the IR illumination is projected directly from the LED onto the phototransistor receiver. As drug formulation passes through this projected “beam”, the particles cast shadows onto the receiver, thereby reducing the amount of received light. This reduction in received light can be converted into an electronic signal and processed in a similar manner as that used for the reflective mode of operation, with the exception that the signal is effectively inverted; that is, a maximum signal level indicates no formulation is present, and a minimum signal indicates that a large amount of formulation is present.

To reduce the complexity of component integration and cost of the inhaler 100, a preferred embodiment utilizes an optical sensor 113 that combines both the transmitter and receiver into a single package. Both reflective mode and transmissive mode sensors are available in this integrated form, as would be known and understood by a person having ordinary skill in the art. However, there may be advantages to using individual components for the transmitter and receiver, primarily lower component cost. Separate transmitter and receiver components can also be arranged for either reflective-mode or transmissive-mode operation.

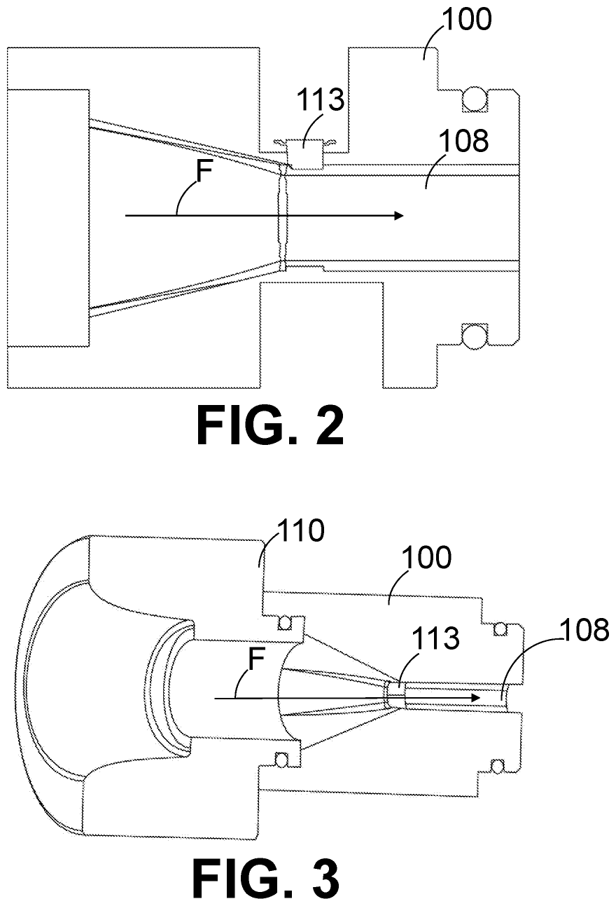

FIG. 4 depicts an exemplary circuit diagram of an optical sensor signal conditioning circuit, in accordance with one or more embodiments. As shown in FIG. 4, the optical sensor signal conditioning circuit 156 may receive and condition optical sensor 113 signals for input into controller 114. The signal conditioning circuit 156 may include the following functional blocks, embodied as subcircuits of the signal conditioning circuit 156:

(1) Sensor and sensor supply circuit, comprised of a DC voltage and decoupling capacitor C5 to supply the phototransistor receiver; and Q1, R1 and R17 to maintain a constant current flowing through the sensor LED, where the LED and phototransistor receiver comprise the optical sensor U2.

(2) Reference voltage circuit, comprised of U3, R13 and C8, which supplies a stable, regulated reference voltage to the LED supply circuit and offset control circuit (4).

(3) Log transimpedance amplifier, comprised of U1A, D3 and C1, which converts the phototransistor receiver current to a voltage proportional to the logarithm of the current. The log amplifier is used to improve amplifier performance by applying non-linear gain to the relatively small signals produced by the optical sensor.

(4) Offset control circuit, comprised of U1D, D1, D2, R9, R14, R10, R11, R12, and C4, which supplies an offset to the log transimpedance amplifier input in order to maintain a constant DC voltage level output from the optical sensor when no powder is present.

(5) Voltage gain stage and low-pass filter, distributed across two inverting amplifier stages, the first stage comprised of U1B, R3, R15, R4, C2, and the second stage comprised of U1C, R16, R5, R6, R7, R8, and C3. The gain stage amplifies the sensor output signal with a high gain (about 484 V/V) necessary to scale the signal to levels appropriate for sampling with a microcontroller-based or data acquisition system-based analog-to-digital converter.

In a preferred embodiment, conditioning circuit 156 may be integrated into the controller 114 of inhaler 100 either as a fully integrated embodiment, or as a separate module.

Inhalation Detection and Triggering of the Vibrator Element

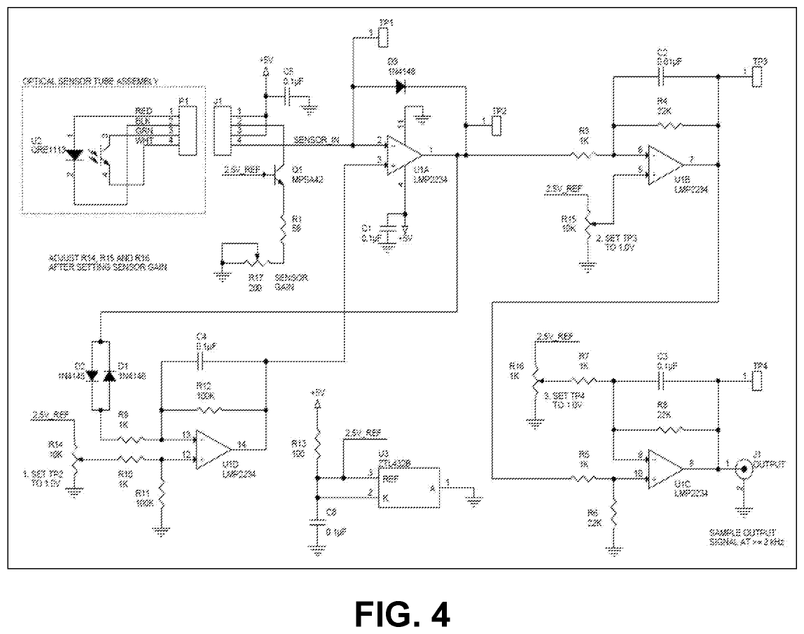

FIG. 5 illustrates various functional components and operation of controller 114. As will be understood by those skilled in the art, although the functional components shown in FIG. 5 are directed to a digital embodiment, it will be appreciated that the components of FIG. 5 may be realized in an analog embodiment.

In one embodiment, controller 114 may include a microcontroller 150 for controlling the power supplied to transducer 102 based on the user's breath cycle and amount of powder medication delivered to the user. In a preferred embodiment, controller 114 may determine the user's breath cycle based on the signals received from inhalation sensor 112. In one embodiment, after the inhaler 100 is turned on, the pressure in air flow conduit 108 may be monitored by inhalation sensor 112 to determine when the user starts breathing. For example, microcontroller 150 may determine whether the user is breathing by calculating the rate of change of pressure within air flow conduit 108. The rate of change of pressure may then compared to predetermined upper and lower limits to ensure an appropriate rate of change has occurred. These upper and lower limits are utilized to reject ambient pressure disturbances in the environment, such as sudden changes in altitude, use of the tidal inhaler in a moving vehicle, opening or closing of doors, fast-moving weather systems, etc. that could results in false triggers due to the high sensitivity of the inhalation sensor. When the rate of change is between the predetermined upper and lower limit, the start of an inhalation of a breath cycle has been detected.

In some embodiments, once the start of inhalation has been detected, microcontroller 150 may accumulate pressure values scaled to volumetric flow rate units to calculate an inhalation volume. As breathing continues, the accumulation of scaled pressure values may be stopped in response to the pressure values crossing the zero point into a positive range where exhalation begins. In one embodiment, microcontroller 150 may compare the inhalation volume to a predetermined threshold to determine if the detected volumetric value is an appropriate inhalation volume. If the inhalation volume exceeds the predetermined threshold, the microcontroller 150 may detect a start of inhalation for a next breath cycle of the user. If the inhalation volume does not exceed the predetermined threshold, the current breath is ignored and determination of the inhalation volume for the first breath cycle of a user is repeated. In a preferred embodiment, microcontroller 150 may continuously monitor the signals received from inhalation sensor 112 to determine the user's breath cycle.

In some embodiments, when the start of the next inhalation is detected as an appropriate rate of change of pressure, and the relative pressure exceeds a predetermined triggering threshold, microcontroller 150 may generate a dosing trigger. In response to the dosing trigger being generated in a second breath cycle, microcontroller 150 may advance the drug strip into position relative to the dosing chamber 118. In response to the dosing trigger being generated for any subsequent breath cycle, microcontroller 150 may activate piezoelectric element 102 for a predetermined amount of time to deliver the drug to the user. In some embodiments, the dosing scheme may activate the piezoelectric element 102 for a predetermined duration of time. For example, the dosing trigger may activate the piezoelectric element 102 for about 100 milliseconds for the third through sixth breath cycles and may activate the piezoelectric element 102 for about 300 milliseconds for the seventh through tenth breath cycles (a total activation time of about 1.6 seconds). It should be appreciated that the number of breath cycles and the predetermined duration of time for the dosing scheme are not limiting and may vary based on the characteristics of the drug and/or user.

It should be appreciated that the dosing session may be repeated for one or more subsequent breath cycles to ensure that the entire dose of powder medication is delivered. As described in greater detail below, controller 114 may also control activation of transducer 102 based on the amount of powder medication that has been delivered to the user. It will be appreciated that the number of breath cycles and the predetermined duration of time for a dosing session are not limiting and may vary based on the characteristics of the drug and/or user.

Optical Dose Sensing

In one embodiment, microcontroller 150 may control the power supplied to transducer 102 based on the amount of powder medication delivered to the patient. For example, microcontroller 150 may determine the amount of powder medication that has been delivered to the user based on the signal received from optical sensor 113 and an estimation formula stored in memory 152. In some embodiment, microcontroller 150 may control activation of transducer 102 until the estimated delivered amount of powder medication reaches a predetermined dosing threshold thus completing the dosing session.

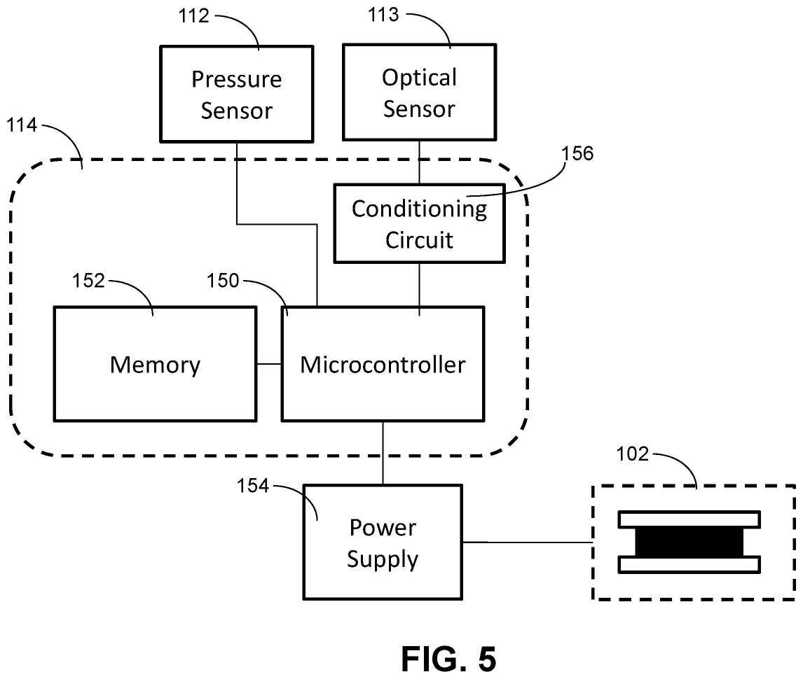

In some embodiments, controller 114 may activate transducer 102 at the start of each inhalation of the user for a series of breath cycles until all the powder medication for a dosing session has been delivered into the user's inspiratory flow. In some embodiments, controller 114 may apply digital signal processing techniques to extract various attributes of the optical sensor 113 signal to estimate the amount of drug formulation that has passed into the user's inspiratory flow. For example, various signal attributes may be used to estimate the amount of formulation delivered including peak signal with respect to time, signal rise and fall times, spectral content and area-under-the-curve (AUC) obtained, for example, by integrating the signal with respect to time and scaling the resulting AUC value with a calibration factor that converts it to actual mass flow. FIG. 6 shows a graph depicting an exemplary optical sensor output signal and area-under-the-curve calculated from the output signal, in accordance with one or more embodiments. In particular, FIG. 6 depicts an exemplary optical sensor output (lower traces) as six shots of powder medication are being delivered by the inhaler. The high trace is the area-under-the curve (AUC) calculated from the calculated sampled output which may be utilized to determine the total amount of powder medication delivered to the user.

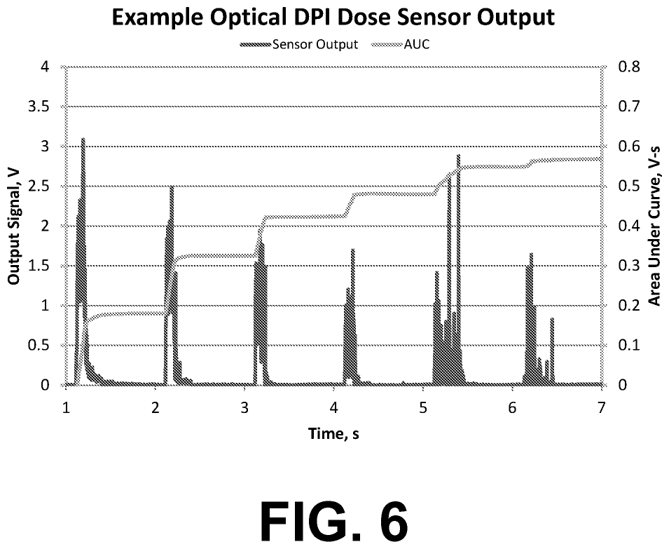

As described above, controller 114 may apply a digital signal processing algorithm to the optical sensor 113 signals to estimate the amount of drug formulation that has passed into the user's inspiratory flow. It has been observed during the use of the inhaler that, depending on the drug powder formulation, finer particles have a tendency to be ejected from the dose chamber early in the dose, whereas larger particles are ejected more slowly and sporadically as the dose chamber is emptied. This may be confirmed through the use of a laser-based particle size analyzer, such as the Sympatec HELOS with INHALER test fixture designed to measure particle size distribution of the dry powder emitted from dry powder inhalers. FIG. 7 shows a graph depicting an output of a particle size analyzer for a single dosing sequence between a second dose shot and sixth dose shot, in accordance with one or more embodiments. In particular, FIG. 7 shows the that the particle size distribution from the inhaler loaded with Respitose (ML-001 lactose) is skewed toward smaller particles for an early dosing shot, then as the shot count within the dose progresses, the distribution shifts toward larger particles.

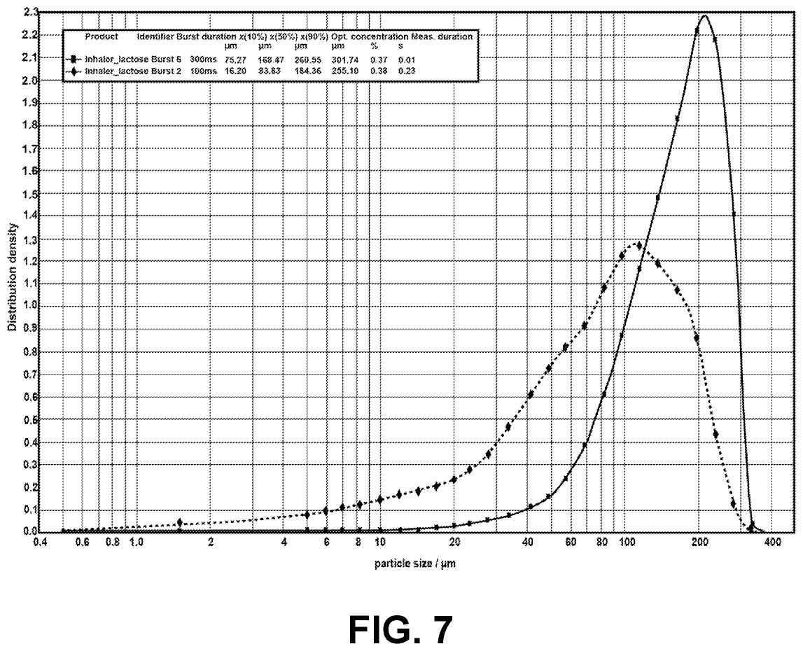

The shift in particle size distribution may also be observed in the output signal of the optical sensor 113, as illustrated in FIG. 8. As shown in FIG. 8, optical sensor output (a) depicts an output for finer particles whereas optical sensor output (b) depicts an output for coarser particles. Optical sensor signals captured during the first of a series of dosing shots contain a larger area under the curve below the high frequency content of the signal, where this area contains essentially no high frequency signal components generated by the sensor. As the dosing shots progress within a single dosing sequence, the clear area under the curve decreases to the point where only high frequency signal content is seen. It was reasoned that a cloud of fine particles would reflect the sensor transmitter's light back to the sensor receiver with a higher intensity as a more diffuse signal resulting in a stronger, low frequency signal response similar to the manner in which a car's headlights are reflected from a heavy fog making it difficult to see other objects whereas fewer larger particles would be seen as individual signal features, or “spikes” as the particles moved past the sensor as they are entrained in the air flow.

One of the challenges in estimating the mass of powder delivered from these sensor output signals is that the finer particles, while producing a larger signal, may contain less mass than fewer coarse particles, so a simple calculation of Area-Under-the-Curve (AUC) may potentially lead to large errors in estimated mass. For this reason, a more sophisticated signal processing algorithm is required to extract the appropriate information from the different signals in order to more accurately account for the differences in the particle size content in each case.

The below algorithm calculates two components of the sampled signal as follows. Area-Under-the-Curve, AUC, is approximated from a left Riemann Sum as:

$$AUC = {\sum }_{k = 0}^{n - 1} {V}_{k} \times Δ t$$

in units of [volt-second] and where Vₖ is the sensor output voltage sample, and Δt is the signal sampling interval. Other formulas may be used to calculate or approximate the area-under-the-curve. The Root Mean Square, or RMS, component is calculated as:

$$RMS = A \times \sqrt{{\sum }_{k = 0}^{n - 1} {V}_{k}^{2}}$$

in units of [volt] where A represents a normalizing scale factor, and the estimated mass, Mₑₛₜ, is then calculated as:

Mₑₛₜ=C×(a×AUC+b×RMS)+i

where a and are constants used to adjust the relative weights of each of the two factors, C is a scale factor relating the estimated mass value to actual mass units derived from the slope of the linear regression model, and i is the y-intercept derived from the linear regression model.

In order to determine the values of the scale factors, the amount of powder delivered by the inhaler through the optical sensor was determined gravimetrically so that the processed optical sensor output could be compared against the known mass of delivered powder. The gravimetric method involved weighing foil blisters containing powder, or molded dose chambers manually loaded with powder, before and after delivering the powder using the active inhaler device, and then subtracting the final value from the initial value to determine net mass of powder delivered.

For each test sample, the time-domain signal output from the optical sensor system (sampled at 2,000 samples per second) was captured using a National Instruments LabView-based data acquisition system. The AUC and RMS values were calculated for each sample according to the above equations. The values of delivered mass determined gravimetrically were placed in a table alongside the calculated AUC and RMS values such that a simple linear regression could be performed in which delivered mass was the dependent variable, y, and the weighted sum of ADC and RMS values calculated for each sample was the independent value, x. A subset of the data collected from the experiments is shown in the table below, where 0.5 was used for the weighting constants a and b.

| | | Cal- | Cal- | Weighted | Weighted | |
|---|---|---|---|---|---|---|
| Sample | Deliv. | culated | culated | AUC | RMS | Weighted |
| No. | Wt. | AUC | RMS | (a = 0.5) | (b = 0.5) | Sum |
| 1 | 1.40 | 0.570 | 12147 | 0.570 | 0.759 | 0.665 |
| 2 | 1.29 | 0.528 | 11399 | 0.528 | 0.712 | 0.670 |
| 3 | 1.46 | 0.566 | 11068 | 0.566 | 0.692 | 0.629 |
| 4 | 1.49 | 0.527 | 10547 | 0.527 | 0.659 | 0.593 |
| 5 | 1.91 | 0.677 | 13162 | 0.677 | 0.823 | 0.750 |
| 6 | 1.69 | 0.874 | 8904 | 0.874 | 0.557 | 0.715 |
| 7 | 2.03 | 0.933 | 9870 | 0.933 | 0.617 | 0.775 |
| 8 | 2.19 | 0.966 | 11328 | 0.966 | 0.708 | 0.837 |
| 9 | 2.23 | 0.819 | 12703 | 0.819 | 0.794 | 0.806 |
| 10 | 2.03 | 0.555 | 15041 | 0.555 | 0.940 | 0.748 |
| 11 | 0.55 | 0.313 | 4527 | 0.313 | 0.283 | 0.298 |
| 12 | 0.47 | 0.312 | 3471 | 0.312 | 0.217 | 0.264 |
| 13 | 0.55 | 0.272 | 3539 | 0.272 | 0.221 | 0.247 |
| 14 | 0.52 | 0.253 | 3263 | 0.253 | 0.204 | 0.229 |
| 15 | 0.59 | 0.228 | 3405 | 0.228 | 0.213 | 0.220 |
| 16 | 0.48 | 0.255 | 3895 | 0.255 | 0.243 | 0.249 |
| 17 | 0.52 | 0.263 | 3781 | 0.263 | 0.236 | 0.249 |
| 18 | 0.70 | 0.329 | 4839 | 0.329 | 0.302 | 0.316 |
| 19 | 0.53 | 0.260 | 4462 | 0.260 | 0.279 | 0.269 |
| 20 | 0.78 | 0.352 | 5968 | 0.352 | 0.373 | 0.362 |
| 21 | 1.71 | 0.557 | 8576 | 0.557 | 0.536 | 0.546 |
| 22 | 2.56 | 0.670 | 13461 | 0.670 | 0.841 | 0.756 |
| 23 | 2.19 | 0.485 | 17041 | 0.485 | 1.065 | 0.775 |
| 24 | 1.93 | 0.322 | 16753 | 0.322 | 1.047 | 0.685 |
| 25 | 1.12 | 0.137 | 12557 | 0.137 | 0.785 | 0.461 |

The normalizing scale factor for the RMS value, A, was determined empirically by dividing each of the calculated RMS values by the maximum RAIS value. This process was repeated for each calibration data set that was collected, and it was found that the value of A was relatively constant across the data sets, so the average value was rounded to a value of 16000, which was used in determining the mass scale factor, C and the weighting constants a and b.

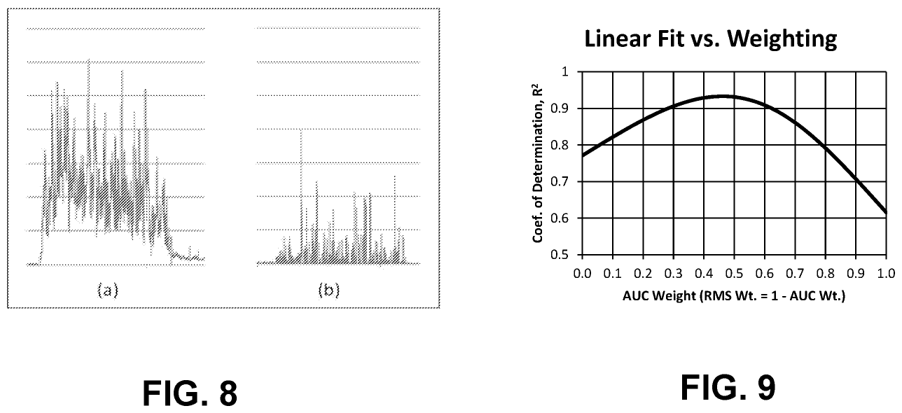

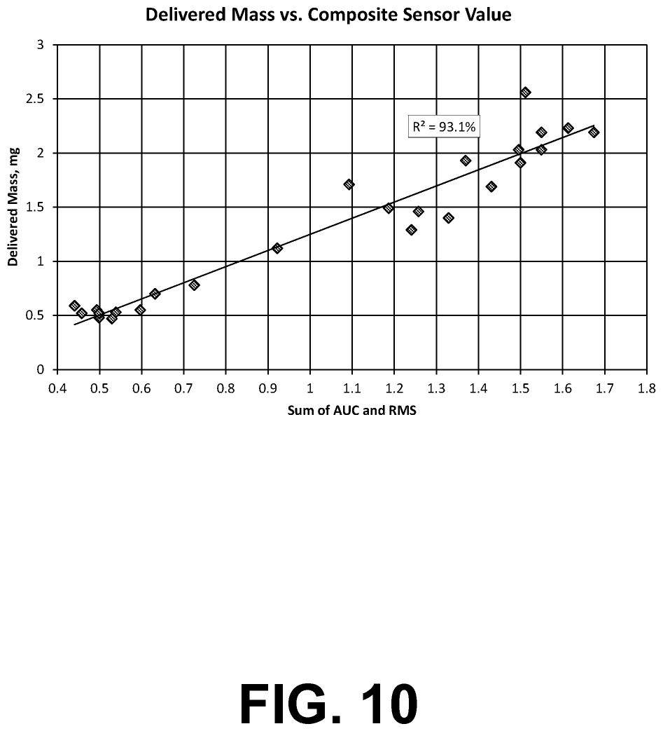

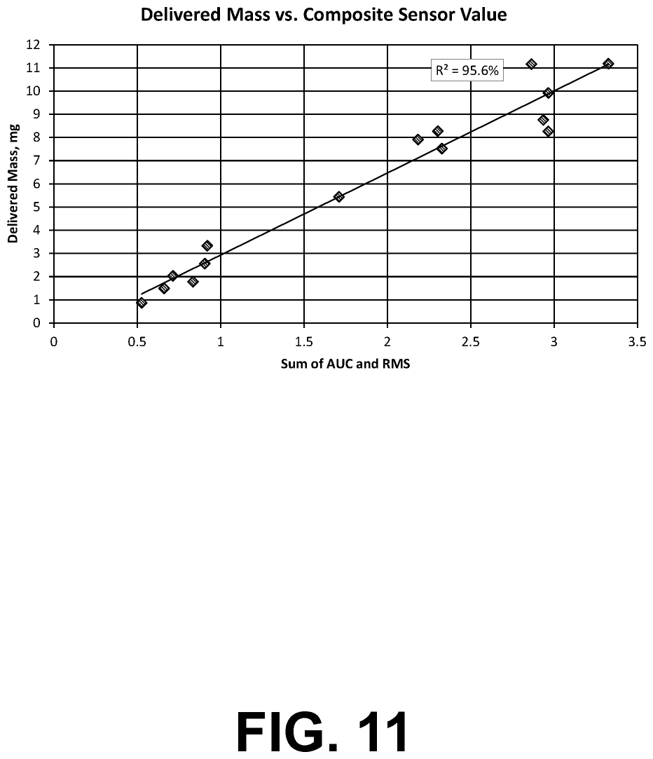

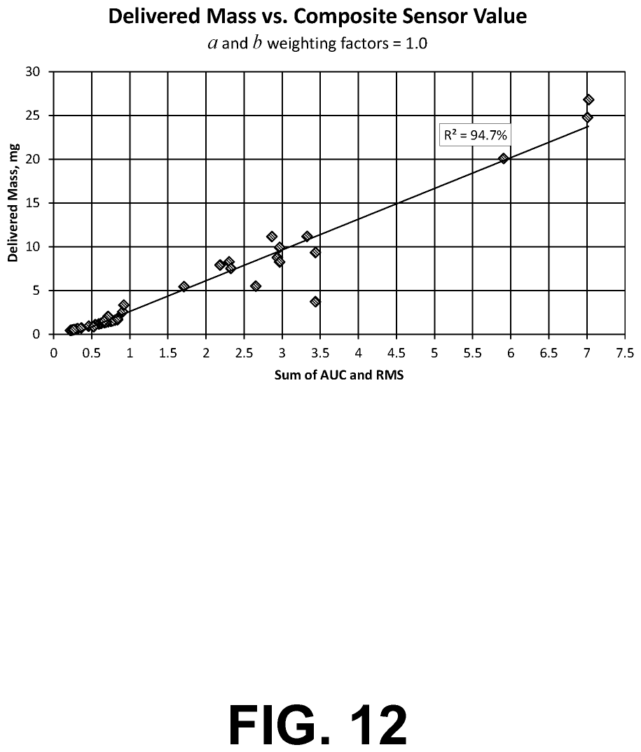

Coefficient of determination, R², was plotted against weight values from 0 to 1 (α=0.0, 0.1, 0.2, . . . 1.0 while, correspondingly, b=1.0, 0.9, 0.8, . . . 0.0) for each of the two variables. The peak of this curve (shown in FIG. 9) determines the best fit of the line modeling a linear relationship between delivered mass and the resulting weighted sum of AUC and RMS. For each of the calibration data sets, the peak of this curve occurred at about 0.5, indicating that equal weights of the AUC and RMS values resulted in the most accurate prediction of delivered powder mass. Since equal weighting of both the AUC and RMS values resulted in the best linear fit, a value of 1.0 was used for both weighting constants a and b in FIG. 10, FIG. 11, and FIG. 12.

Using a value of 1.0 for both weighting factors a and b, the simple linear regression model yields the following values:

C(slope)=3.51

i(y intercept)=−0.90

thus the formula for estimating delivered mass of powder medication in mg is:

Mₑₛₜ=3.51×(AUC+RMS)−0.90

It will be appreciated by persons having ordinary skill in the art that the parameters used in this model are valid for the optical sensor embodiment described by FIGS. 2 and 3, and that the parameters could vary for other optical sensors. There are a number of factors that may affect the transfer function of the sensor system including, but not limited to: sensor amplifier gain and transfer function (for example, a non-linear amplifier stage was used in this embodiment), optical sensor gain, width of sensing channel, reflectance of material used for the sensor tube, ambient IR interference, operating temperature (temperature compensation could be added to the design to improve accuracy), infrared absorption characteristics of the powder being measured, reflectivity of the powder being measured, particle size characteristics of the powder being measured, and the rate at which particles move by the sensor, which is determined by the air flow rate. Those skilled in the art, using the guidelines provided herein, will be capable of developing a suitable model for various optical sensors.

In a preferred embodiment, controller 114 utilizes the signals received from optical sensor 113 and the formula for estimating delivered mass of powder medication stored in memory 152 to estimate the mass of powder medication delivered to a patient during an inhalation. For example, for each inhalation, the amount of powder medication delivered to the user is estimated. After each inhalation, the estimation of powder medication delivered is summed with the estimation from each previous inhalation and compared to a predetermined dosing threshold stored in memory. Thus, the total estimation of powder medication delivered to the user is determined. If the total estimation of powder medication delivered does not reach the predetermined dosing threshold, controller 114 can activate transducer 102 during the next inhalation to deliver additional powder medication. If the total estimation of powder medication delivered reaches the predetermined dosing threshold, controller 114 communicates to the user through the inhaler's user interface that the dosing session is complete, and/or de-activates transducer 102 so that additional medication is not delivered during subsequent inhalations.

As described above, controller 114 may utilize information about the user's breath cycle (based on the signal received from inhalation sensor 112) with the optical sensor information (based on the signal received from optical sensor 113) to determine that the powder medication was released during optimal air flow conditions as the patient is inhaling. This information may be presented to the patient during and/or immediately after a dose is taken via the inhaler's user interface to allow the patient to confirm that each dose was properly taken. In the event that the inhaler erroneously releases formulation during sub-optimal air flow conditions such as exhalation of the breath cycle, the optical sensor information combined with the air flow information from the inhaler's breathing sensor results in an error condition that can be communicated to the user via the inhaler's user interface, allowing the patient to take corrective action if necessary.

Exemplary Flowcharts

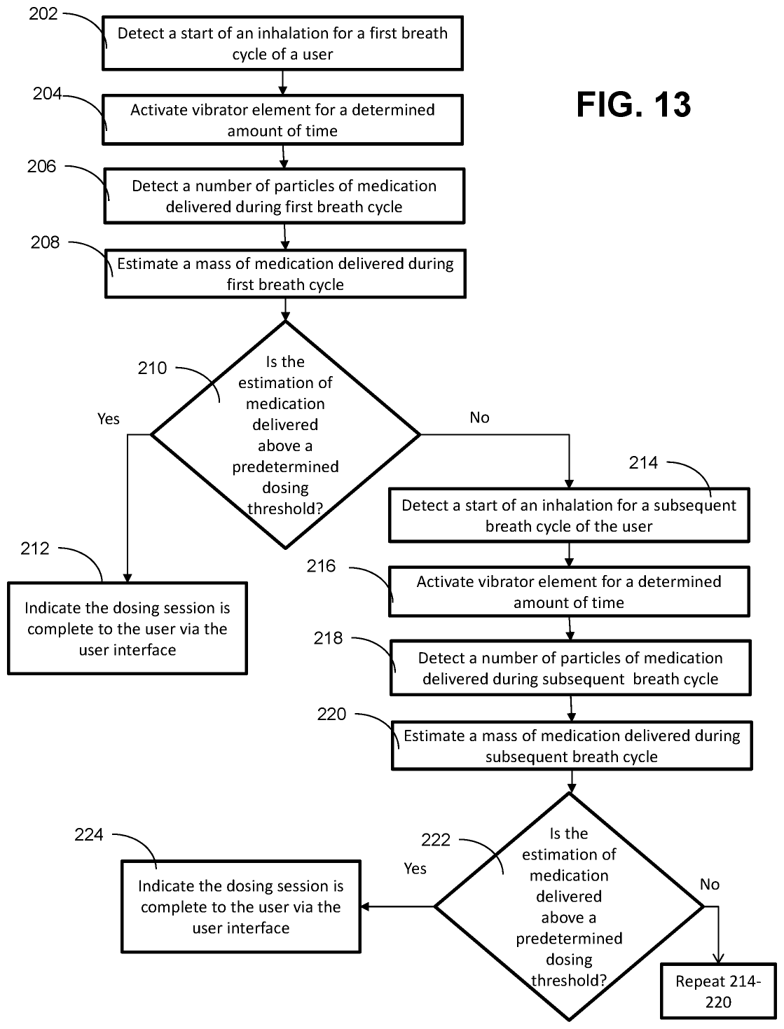

FIG. 13 depicts a flowchart of a method 200 for delivering a dose of a drug with an inhaler, in accordance with one or more embodiments.

In an operation 202, a start of an inhalation of a first breath cycle of a user is detected. As an example, after the inhaler is turned on, the pressure in the flow channel is monitored to determine when the user starts an inhalation. This is determined by calculating the rate of change of pressure within the flow channel. The rate of change of pressure is then compared to predetermined upper and lower limits to ensure an appropriate rate of change has occurred. If the rate of change is not within the predetermined upper and lower limits, the current breath cycle is ignored and detection of the start of an inhalation for the first breath cycle of the user is repeated.

In an operation 204, the vibrator element is activated for a predetermined amount of time in response to the start of inhalation for the first breath cycle being detected. For example, the dosing trigger may activate the piezoelectric element 90 for about 100 milliseconds for the third through sixth breath cycles and the dosing trigger may activate the piezoelectric element 90 for about 300 milliseconds for the seventh through tenth breath cycles (a total activation time of about 1.6 seconds). It will be appreciated that the number of breath cycles and the predetermined duration of time for the dosing scheme are not limiting and may vary based on the characteristics of the drug and/or user. For example, the dosing trigger may activate the piezoelectric element for any where from about 25 to about 250, or from about 50 to about 200, or from about 65 to about 145, or from about 75 to about 125, or about 100 milliseconds for the third through sixth breath cycles, and the dosing trigger may activate the piezoelectric element for anywhere from about 125 to about 650, or from about 175 to about 500, or from about 225 to about 400, or from about 250 to about 350, or about 300 milliseconds for the seventh through tenth breath cycles, or any values therebetween.

In an operation 206, a number of particles of powder medication being delivered to the user during the first breath cycle is detected. For example, and optical sensor may be positioned on the inner surface of conduit of inhaler to sense the passing of particles of powder medication by the sensor through air stream F. It should be appreciated that optical sensor may be configured for either reflective-mode or transmissive mode operation to sense particle of powder medication.

In an operation 208, a mass of powder medication delivered to the user during the first breath cycle is estimated. For example, the mass of powder medication delivered is calculated from signals received from the optical sensor and the formula for estimating delivered mass of powder medication stored in memory.

In an operation 210, the estimated mass of powder medication delivered is compared to a predetermined dosing threshold. For example, a predetermined dosing threshold for the total amount of medication to be delivered it utilized to determine whether the dosing session is complete.

In response to the estimated mass of powder medication being equal to or above the predetermined dosing threshold, the user is indicated through the user interface that the dosing session is complete in operation 212.

In response to the estimated mass of powder medication being less than the predetermined dosing threshold, a start of an inhalation of a subsequent breath cycle of a user is detected in operation 214, similar to operation 202.

In an operation 216, the piezoelectric element is activated for a predetermined amount of time in response to the start of inhalation for the subsequent breath cycle being detected, similar to operation 204.

In an operation 218, a number of particles of powder medication being delivered to the user during the subsequent breath cycle is detected, similar to operation 206.

In an operation 220, a mass of powder medication delivered to the user during the subsequent breath cycle is estimated, similar to operation 208.

In an operation 222, the estimated mass of powder medication delivered is compared to a predetermined dosing threshold, similar to operation 210.

In response to the estimated mass of powder medication being equal to or above the predetermined dosing threshold, the user is indicated through the user interface that the dosing session is complete in operation 224, similar to operation 212.

In response to the estimated mass of powder medication being less than the predetermined dosing threshold, repeat operations 214-220.

It will be appreciated and understood by those having ordinary skill in the art that operations 214 through 220 may be repeated for one or more subsequent breath cycles to ensure that the entire that the correct amount of powder medications for the dosing session was delivered to the user.

Although the embodiments have been described in detail for the purpose of illustration based on what is currently considered to be the most practical and preferred embodiments, it is to be understood that such detail is solely for that purpose and that the embodiments are not limited to the disclosed preferred features, but, on the contrary, is intended to cover modifications and equivalent arrangements that are within the scope of the appended claims. For example, it is to be understood that the features disclosed herein contemplate that, to the extent possible, one or more features of any embodiment can be combined with one or more features of any other embodiment.

What is claimed is:

1. A dry powder inhaler comprising:
a first chamber configured to hold a dry powder and a gas;
a second chamber connected to the first chamber by at least one passageway configured to receive an aerosolized form of the dry powder from the first chamber and configured to deliver the aerosolized dry powder to a user;
at least one optical sensor configured to monitor particles of dry powder passing the optical sensor in the second chamber;
a vibrator coupled to the first chamber configured to aerosolize the dry powder and cause the aerosolized powder to move through the at least one passageway thereby delivering the dry powder from the first chamber to the second chamber as an aerosolized dry powder; and
a vibrator control unit configured to control operation of the vibrator based on an amount of particles in the second chamber passing the at least one optical sensor.

2. The inhaler of claim 1, wherein the vibrator control unit is further configured to:
estimate an amount of dry powder delivered based on the amount of particles in the second chamber passing the at least one optical sensor.

3. The inhaler of claim 2, wherein the vibrator control unit is further configured to:
compare the estimated amount of dry powder delivered to a predetermined dosing threshold;
in response to the estimated amount of dry powder delivered to the user being greater or equal to the predetermined dosing threshold, indicate to the user that dosing is complete.

4. The inhaler of claim 1, wherein the dry powder inhaler further includes:
a inhalation sensor to monitor the pressure in the second chamber; and wherein the vibrator control unit is further configured to:
determine a user's breath cycle based on the monitored pressure in the second chamber.

5. The inhaler of claim 4, wherein the vibrator control unit is further configured to:
in response to the estimated amount of dry powder delivered to the user being less than the predetermined dosing threshold, activate the vibrator for a predetermined time for a next inhalation of the user's breath cycle.

6. The inhaler of claim 2, wherein the estimation of an amount of dry powder delivered is based on output signals received from the at least one optical sensor.

7. The inhaler of claim 1, wherein the at least one optical sensor operates in a reflective-mode such that light transmitted from a transmitter is reflected off aerosolized powder and is received by a receiver.

8. The inhaler of claim 1, wherein the at least one optical sensor operates in a transmissive-mode such that aerosolized powder blocks the amount of light, transmitted from a transmitter, that is received by a receiver.

9. The inhaler of claim 1, where in the at east optical sensor combines both a transmitter and a receiver in a single package.

10. A method for delivering a dose of a drug with an inhaler, the method comprising:
holding a dry powder and a gas in a first chamber;
receiving an aerosolized form of the dry powder in a second chamber connected to the first chamber;
delivering the aerosolized dry powder in the second chamber to a user;
monitoring particles of dry powder passing by at least one optical sensor positioned in the second chamber;
controlling operation of the vibrator based on an amount of particles in the second chamber passing the at least one optical sensor; and
aerosolizing the dry powder with a vibrator coupled to the first chamber, wherein the vibrator causes the aerosolized powder to move through the at least one passageway thereby delivering the dry powder from the first chamber to the second chamber as an aerosolized dry powder.

11. The method of claim 10, wherein the method further includes:
estimating an amount of dry powder delivered based on the amount of particles in the second chamber passing the at least one optical sensor.

12. The method of claim 11, wherein the method further includes:
comparing the estimated amount of powder delivered to a predetermined dosing threshold; and
in response to the estimated amount of dry powder delivered to the user being greater or equal to the predetermined dosing threshold, indicating to the user that dosing is complete.

13. The method of claim 10, wherein the method further includes:
monitoring the pressure in the second chamber with a inhalation sensor; and
determining a user's breath cycle based on the monitored pressure in the second chamber.

14. The method of claim 13, wherein the method further includes:
in response to the estimated amount of dry powder delivered to the user being less than the predetermined dosing threshold, activating the vibrator for a predetermined time for a next inhalation of the user's breath cycle.

15. The method of claim 11, wherein the estimation of an amount of dry powder delivered is based on output signals received from the at least one optical sensor.

16. The method of claim 10, wherein the at least one optical sensor operates in a reflective-mode such that light transmitted from a transmitter is reflected off aerosolized powder and is received by a receiver.

17. The method of claim 10, wherein the at least one optical sensor operates in a transmissive-mode such that aerosolized powder blocks the amount of light, transmitted from a transmitter, that is received by a receiver.

18. The method of claim 10, where in the at least optical sensor combines both a transmitter and a receiver in a single package.
